(19) United States
(12) Patent Application Publication
(10) Pub. No.: US 2021/0015934 A1
(43) Pub. Date: Jan. 21, 2021

(54) Method of Treating Parkinson's Disease

(71) Applicant: Venturis Therapeutics, Inc., Dallas, TX (US)

(72) Inventors: Laurence R. Meyerson, Henderson, NV (US); Adam Nedella, Dallas, TX (US)

(21) Appl. No.: 16/932,113

(22) Filed: Jul. 17, 2020

Related U.S. Application Data
(60) Provisional application No. 62/875,668, filed on Jul. 18, 2019.

(51) Int. Cl. A61K 47/64; A61K 38/18; A61K 31/727; A61B 5/00; A61K 35/545; A61K 9/00; A61B 5/055; A61K 35/28

(52) U.S. Cl. CPC A61K 47/646; A61K 38/1825; A61K 31/727; A61B 5/0042; A61K 35/28; A61K 35/545; A61K 9/0019; A61B 5/055; A61B 5/0036

(57) ABSTRACT

The present invention includes a method of treating Parkinson's disease in a patient comprising: administering a therapeutically effective amount of a FGF-1¹⁵⁻¹⁵⁵ and mutants thereof conjugated to a heparin (FGF-1), which is an amount sufficient to reduce or eliminate one or more symptoms associated with Parkinson's disease.

### CROSS-REFERENCE TO RELATED APPLICATIONS

This application claims priority to U.S. Provisional Application Ser. No. 62/875,668, filed Jul. 18, 2019, the entire contents of which are incorporated herein by reference.

### TECHNICAL FIELD OF THE INVENTION

The present invention relates in general to the field of compositions and methods for treating Parkinson's disease.

### STATEMENT OF FEDERALLY FUNDED RESEARCH

None.

### INCORPORATION-BY-REFERENCE OF MATERIALS FILED ON COMPACT DISC

The present application includes a Sequence Listing which has been submitted in ASCII format via EFS-Web and is hereby incorporated by reference in its entirety. Said ASCII copy, created on Jul. 17, 2020, is named VENT1009_SeqList.txt and is 3, kilobytes in size.

### BACKGROUND OF THE INVENTION

Without limiting the scope of the invention, its background is described in connection with treatments for Parkinson's disease.

U.S. Pat. No. 9,006,177, issued to Barawkar, et al., entitled, “Fused tricyclic compounds as adenosine receptor antagonist” is directed to tricyclic compounds or its tautomers, polymorphs, stereoisomers, prodrugs, solvate or a pharmaceutically acceptable salts, or pharmaceutical compositions containing them and methods of treating conditions and diseases that are mediated by thereof as A2A adenosine receptor antagonists. These tricyclic compounds are said to be useful in the treatment, prevention or suppression of diseases and disorders that may be susceptible to improvement by the mediation of adenosine A2A receptor, including, but not limited to: Parkinson's disease, restless leg syndrome, Alzheimer's disease, neurodegenerative disorder, inflammation, wound healing, dermal fibrosis, nocturnal myoclonus, cerebral ischemia, myocardial ischemia, Huntington's disease, multiple system atrophy, cortico-basal degeneration, Wilson's disease or other disorders of basal ganglia which results in dyskinesias, post-traumatic stress disorder, hepatic cirrhosis, sepsis, spinal cord injury, retinopathy, hypertension, social memory impairment, depression, neuroprotection, narcolepsy or other sleep related disorders, attention deficit hyperactivity disorder, drug addiction, post traumatic stress disorder and vascular injury and the like.

U.S. Patent Publication No. 20140243293, filed by Bose, et al., and entitled “Phosphaplatins as Neuroprotective Agents” is said to be directed to agents for the treatment of neurodegenerative diseases comprising one or more isolated phosphate complexes of platinum and methods of uses thereof for treating neurodegenerative diseases including amyotrophic lateral sclerosis, Alzheimer's disease, stroke, epilepsy, Parkinson's, Huntington's disease and diabetes associated peripheral neuropathy. The phosphaplatins are anti-angiogenic compositions useful for inhibiting angiogenesis related to age-related macular degeneration, diabetic retinopathy and tumor-associated angiogenesis.

Kryzhanovsky, G. N., et al., in a publication entitled “Effects of fibroblast growth factors on MPTP-induced parkinsonian syndrome in mice”, Pathophysiology 4 (1997) 59-67, compared the effects of acidic fibroblast growth factor (FGF-1) and basic fibroblast growth factor (FGF-2) provided to a mouse intranasally, the mice having been treated with the dopaminergic neurotoxin 1-methyl-4-phenyl-1,2,3,6-tetrahydropyridine (MPTP). The authors indicate that the MPTP may be causing a disruption in the blood brain barrier, thereby allowing the intranasally administered wild-type FGF-1 to reach the brain. The authors found that intranasal FGF-1 was only able to reduce oligokinesia, tremors and muscular rigidity, while FGF-2 acted to predominantly prevent weight loss and increased survival.

Wei, et al., in a publication entitled “Fibroblast growth factor 1 attenuates 6-hydroxydopamine-induced neurotoxicity: an in vitro and in vivo investigation in experimental models of Parkinson's disease”, Am J Transl Res 2014; 6(6):664-677, injected FGF-1 via tail vein in a 6-hydroxydopamine (6-OHDA) toxicity model system for PD. These authors found some reduction in PD symptoms by intracerebrally injecting wild-type FGF-1 unilaterally into the striatum on the right of the brain at a constant rate. These inventors found that intracerebrally injected FGF-1 improved the motor function recovery, increased the TH-positive neurons survival, and up-regulated the levels of neurotransmitters in PD rats.

What is needed are compositions and methods for the reduction or elimination of Parkinson's disease symptoms and disease, in particular, the substantial reduction or elimination of bradykinesia, progressive reduction in speed and range of repetitive actions such as voluntary finger-tapping, in combination with one of three other physical signs: muscular (lead-pipe or cogwheel) rigidity, tremor at rest, and postural instability.

### SUMMARY OF THE INVENTION

In one embodiment, the present invention includes a method of treating Parkinson's disease in a patient comprising: administering a therapeutically effective amount of a Fibroblast Growth Factor-1 (FGF-1), or active mutants or variants thereof conjugated to a heparin (FGF-1), which is an amount sufficient to reduce or eliminate one or more symptoms associated with Parkinson's disease. In one aspect, the FGF-1 is selected from 140 or 141 amino acid protein of SEQ ID NO:1, which is the mature form of human, also referred to as: FGF-1, FGF-1¹⁻¹⁴⁰, FGF-1¹⁻¹⁴¹, FGF-1¹⁰⁻¹⁴⁰, FGF-1¹⁰⁻¹⁴¹ FGF-1¹⁻¹⁴⁰, FGF-1¹²⁻¹⁴⁰, FGF-1¹²⁻¹⁴¹, and mature FGF-1 with point mutants including, for example, one or more of the following: K9A, K12V, S17R, N18R, N18K, H21Y, R35E, L44F, A66C, Y94V, N95V, H102Y, F108Y, N114R, N114K, C117V, L133A of SEQ ID NO:1. In another aspect, the FGF-1, active mutants or variants thereof, is formulated for intravenous administration. In another aspect, the method further comprises the steps of: imaging a patient to generate image data; obtaining image data of the patient, the image data including at least a portion of a brain proximal to a basal ganglia or a substantia nigra; and introducing at or about the basal ganglia or the substantia nigra a first amount of the FGF-1, active mutants or variants. In another aspect, the method further comprises introducing a first therapeutically effective amount of at least one member of the group consisting of embryonic stem cells, adult stem cells, stem cells and progenitor cells to a brain proximal to, or into, a basal ganglia or a substantia nigra. In another aspect, the method further comprises injecting the therapeutically effective amount of at least one member of the group consisting of embryonic stem cells, adult stem cells, stem cells and progenitor cells into a portion of a brain proximal to a basal ganglia or a substantia nigra via a percutaneous or intracerebral route. In another aspect, the method further comprises surgically implanting a tissue graft proximate to a portion of a brain proximal to a basal ganglia or a substantia nigra with an amount FGF-1 sufficient to reduce or eliminate the symptoms of PD, wherein the tissue graft comprises at least one member of the group consisting of embryonic stem cells, adult stem cells, stem cells and progenitor cells into the basal ganglia or the substantia nigra after the tissue graft has been surgically implanted. In another aspect, the method further comprises the steps of obtaining non-invasive image data of the subject after treatment with the FGF-1, wherein the non-invasive image data including the at-risk region of a brain, and analyzing the non-invasive image data to identify any improvement in the blood vessel supply to the at-risk region of the brain In another aspect, the FGF-1 further comprises a time release, biodegradable carrier applied at a surgical grafting site proximal to the basal ganglia or the substantia nigra. In another aspect, the FGF-1 is administered from about 0.5 to 1000 μg/kg, or 1 to 500 μg/kg, or 10 to 500 μg/kg, or 50 to 100 μg/kg of the patient's body weight per dose. In another aspect, the FGF-1 is formulated for intramuscularly, intravenously, intracerebrally, or intra-arterially, administration as a solid, gel, liquid, bolus dose, or by infusion from about 0.1 to 100 μg/kg of the patient's body weight, or 0.3 to 30 μg/kg, or 1 to 3 μg/kg of the patient's body weight per dose. In another aspect, the FGF-1 is not administered intranasally, intraperitoneally, or traversing the cranium. In another aspect, the dose of FGF-1 is greater than 100 ng/ml. In another aspect, the FGF-1 reduces Parkinson's progression without triggering or causing angiogenesis. In another aspect, the FGF-1, FGF-1¹⁻¹⁵⁵, FGF-1¹⁻¹⁴¹, or combinations thereof, is provided at 100, 150, 200, 250, 300, 400, 450, 500, 600, 700, 750, 800, 900 and 1,000 ug/kg/hr to improve for metabolites, behavior, and cell survival in PD. In one aspect, the treatment provided least one of: an 80, 85, 90, 95, or 100% percent cell survival; an 80, 85, 90, 95, or 100% reduction in PD behaviors, an 80, 85, 90, 95, or 100% survival of the subject, or any combination thereof.

In another embodiment, the present invention includes a method treating Parkinson's disease in a patient comprising the steps of: imaging a patient to generate image data; obtaining imaging data of at least a portion of a microvasculature region of the patient proximal to a portion of a brain proximal to a basal ganglia or a substantia nigra; assessing the imaging data to quantify localized perfusion of at least a portion of the microvasculature proximal to the basal ganglia or the substantia nigra and identify at least one hypoperfused region of the microvasculature; and treating the at least one hypoperfused region of the microvasculature by introducing a first amount of a compound comprising a Fibroblast Growth Factor-1 (FGF-1), or active mutants or variants, in a scaffold material proximate to the hypoperfused region of the microvasculature at the basal ganglia or the substantia nigra, wherein the FGF-1¹⁵⁻¹⁵⁵ and mutants thereof is administered for extended released. In one aspect, the FGF-1 is selected from 140 or 141 amino acid protein of SEQ ID NO:1, which is the mature form of human, also referred to as: FGF-1¹⁻¹⁴⁰, but also FGF-1¹⁻¹⁴¹, FGF-1¹⁰⁻¹⁴⁰, FGF-1¹⁰⁻¹⁴¹, FGF-1¹⁻¹⁴⁰, FGF-1¹²⁻¹⁴⁰, FGF-1¹²⁻¹⁴¹, and mature FGF-1 with point mutants including, for example, one or more of the following: K9A, K12V, S17R, N18R, N18K, H21Y, R35E, L44F, A66C, Y94V, N95V, H102Y, F108Y, N114R, N114K, C117V, L133A of SEQ ID NO:1. In another aspect, the FGF-1 is formulated for intravenous administration. In another aspect, the method further comprises the steps of imaging a patient to generate image data; obtaining image data of the patient, the image data including at least a portion of a brain proximal to a basal ganglia or a substantia nigra; and introducing at or about the basal ganglia or the substantia nigra a first amount of a FGF-1. In another aspect, the method further comprises introducing a first therapeutically effective amount of at least one member of the group consisting of embryonic stem cells, adult stem cells, stem cells and progenitor cells to a brain proximal to, or into, a basal ganglia or a substantia nigra. In another aspect, the method further comprises injecting the therapeutically effective amount of at least one member of the group consisting of embryonic stem cells, adult stem cells, stem cells and progenitor cells into a portion of a brain proximal to a basal ganglia or a substantia nigra via a percutaneous route. In another aspect, the method further comprises surgically implanting a tissue graft proximate to a portion of a brain proximal to a basal ganglia or a substantia nigra with an amount FGF-1 sufficient to reduce or eliminate the symptoms of PD, wherein the tissue graft comprises at least one member of the group consisting of embryonic stem cells, adult stem cells, stem cells and progenitor cells into the basal ganglia or the substantia nigra after the tissue graft has been surgically implanted. In another aspect, the method further comprises the steps of obtaining non-invasive image data of the subject after treatment with the FGF-1 or active mutants or variants, wherein the non-invasive image data including the at-risk region of a brain, and analyzing the non-invasive image data to identify any improvement in the blood vessel supply to the at-risk region of the brain. In another aspect, the FGF-1 further comprises a time release, biodegradable carrier applied at a surgical grafting site proximal to the basal ganglia or the substantia nigra. In another aspect, the FGF-1 or active mutants or variants is administered from about 0.5 to 1000 μg/kg, or 1 to 500 μg/kg, or 10 to 500 μg/kg, or 50 to 100 μg/kg of the patient's body weight per dose. In another aspect, the FGF-1 is formulated for intramuscularly, intravenously, or intra-arterially, administration as a solid, gel, liquid, bolus dose, or by infusion from about 0.1 to 100 μg/kg of the patient's body weight, or 0.3 to 30 μg/kg, or 1 to 3 μg/kg of the patient's body weight per dose. In another aspect, the FGF-1 is not administered intranasally, intraperitoneally, or traversing the cranium. In another aspect, the dose of FGF-1 is greater than 100 ng/ml. In another aspect, the FGF-1 reduces Parkinson's progression without triggering or causing angiogenesis. In another aspect, the FGF-1, FGF-1₁₋₁₅₅, FGF-1₁₋₁₄₁, or combinations thereof, is provided at 100, 150, 200, 250, 300, 400, 450, 500, 600, 700, 750, 800, 900 and 1,000 ug/kg/hr to improve for metabolites, behavior, and cell survival in PD. In one aspect, the treatment provided least one of: an 80, 85, 90, 95, or 100% percent cell survival; an 80, 85, 90, 95, or 100% reduction in PD behaviors, an 80, 85, 90, 95, or 100% survival of the subject, or any combination thereof.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show a behavioral analysis after induction of PD with MPTP, the mice were administered FGF1 at the indicated doses.

FIGS. 2A to 2C show the monoamine and metabolite levels in the brains of MPTP treated animals. The efficacy of FGF-1 on brain monoamine and metabolite levels that change during treatment with MPTP and are dopamine-metabolic markers of PD.

FIG. 3 shows the numbers of cells in the SNpc were determined by counting the TH positive neurons with and without treatment with FGF-1.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not limit the invention, except as outlined in the claims.

As used herein, a “therapeutically effective amount” refers to an amount of FGF-1 that elicits the biological or medicinal response indicated. This response may occur in a tissue, system, animal or human and includes alleviation of the symptoms of the disease being treated. The exact formulation, route of administration and dosage for the composition and pharmaceutical compositions disclosed herein can be chosen by the individual physician in view of the patient's condition. (See e.g., Fingl et al. 1975, in “The Pharmacological Basis of Therapeutics”, Chapter 1, which is hereby incorporated by reference in its entirety). Therapeutic treatments can be achieved with small molecule organic drugs or biologics, such as proteins. Typically, the dose range of FGF-1 is administered from about 0.5 to 1000 μg/kg, or 1 to 500 μg/kg, or 10 to 500 μg/kg, or 50 to 100 μg/kg of the patient's body weight per dose. The dose of FGF-1 can be administered to the patient in a variety of ways, including but not limited to topically, subcutaneously, intramuscularly, intravenously and/or intra-arterially, as a solid, gel, liquid, bolus dose and/or by infusion from about 0.1 to 100 μg/kg of the patient's body weight, or 0.3 to 30 μg/kg, or 1 to 3 μg/kg of the patient's body weight per dose. To achieve localized targeted dosing, FGF-1 can be introduced or injected either directly into or adjacent to the ischemic and/or damaged tissue region, preferably either into or as near as practical to the region of ischemia/damage. Localized dose ranges can be from 10 ng/cm³ to 1 mg/cm³, or 100 ng/cm³ to 100 μg/cm3 or 1 μg/cm³ to 10 μg/cm³ of target tissue per dose. Local doses can be administered at each ischemic and/or damaged tissue region. The dosage may be a single one or a series of two or more given in the course of one or more days, as is needed by the patient. Where no human dosage is established, a suitable human dosage can be inferred from ED₅₀ or ID₅₀ values, or other appropriate values derived from in vitro or in vivo studies, as qualified by toxicity studies and efficacy studies in animals.

As used herein, the terms “treating,” “treatment,” “therapeutic,” or “therapy” do not necessarily mean total cure or abolition of the disease or condition. Any alleviation of any undesired signs or symptoms of a disease or condition, to any extent, can be considered treatment and/or therapy. It is entirely possible that “treatment” includes a temporary improvement in the symptoms of Parkinson's Disease (PD) that may require repeated treatments over time to continue the regenerative process. Alternatively, the “treatment” may only marginally improve the symptoms of Parkinson's Disease (PD), but still may require the use of additional therapies for the patient to achieve an acceptable relief from symptoms of Parkinson's Disease (PD). In addition, asymptomatic Parkinson's Disease (PD) (including prophylactic treatment) may be the focus of treatment utilizing angiogenesis. Furthermore, treatment may include acts that may worsen the patient's overall balance, feeling of well-being, or appearance. In certain embodiments of the present invention, the amount of the FGF-1 of the present invention used for treatment provides a reduction of greater than 50, 60, 70, 75, 80, 90, 92, 95, 98, or 100% of the symptoms of PD as a results of the treatment.

As used herein, the term “FGF-1” refers to those native FGF-1, FGF-1 mutants or FGF-1 variants of FGF-1 that are still biologically active (that is, they activate FGF-1 receptors), but have a sequence that is altered from the natural FGF-1 that encodes a 154 or 155 amino acid protein of SEQ ID NO:2. The present invention includes the use of FGF-1¹⁵⁻¹⁵⁵ and mutants thereof. The skilled artisan will recognize that the mature form of FGF-1 has the first 14 amino acids cleaved after synthesis and prior to export. For examples, truncated FGF-1 proteins for use with the present invention include: synthetic genes that encode a 140 or 141 amino acid protein of SEQ ID NO:1, which is the mature form of human (i.e., the first 14 amino acids have been cleaved), also referred to as: FGF-1¹⁻¹⁴⁰, FGF-1¹⁻¹⁴¹, FGF-1¹⁰⁻¹⁴⁰, FGF-1¹⁰⁻¹⁴¹, FGF-1¹⁻¹⁴⁰, FGF-1¹²⁻¹⁴⁰, FGF-1¹²⁻¹⁴¹, and mature FGF-1 with point mutations including, for example, one or more of the following: K9A, K12V, S17R, N18R, N18K, H21Y, R35E, L44F, A66C, Y94V, N95V, H102Y, F108Y, N114R, N114K, C117V, L133A of the mature form of the human FGF-1 (FGF-1¹⁵⁻¹⁵⁵) which has the amino acid sequence: MFNLPP GNYKKPKLLY CSNGGHFLRI LPDGTVDGTRDRSDQHIQLQ LSAESVGEVY IKSTETGQYL AMDTDGLLYG SQTPNEECLF LERLEENHYN TYISKKHAEK NWFVGLKKNG SCKRGPRTHY GQKAILFLPL PVSSD (SEQ ID NO:1). The full length human FGF-1 is UniProtKB-P05230 (FGF1_HUMAN), Entrez Gene: 2246) SEQ ID NO: 2 MAEGEITTFT ALTEKF NLPP GNYKKPKLLY CSNGGHFLRI LPDGTVDGTRDRSDQHIQLQ LSAESVGEVY IKSTETGQYL AMDTDGLLYG SQTPNEECLF LERLEENHYN TYISKKHAEK NWFVGLKKNG SCKRGPRTHY GQKAILFLPL PVSSD (SEQ ID NO:2). Mutant FGF-1s with one or more amino acid insertions, deletions or substitutions are introduced by standard genetic engineering techniques, such as site-directed, deletion, and insertion mutagenesis. The wild type FGF-1 three-dimensional conformation is known to be marginally stable with denaturation occurring either at or near physiologic temperature. FGF-1 binding to heparin increases the thermal inactivation temperature by approximately 20° C., thus, in certain embodiments the mutant FGF-1 is combined with heparin. Further, the FGF-1 of the present invention can also be formulated with a therapeutically approved USP heparin, or a mutant heparin. The truncations, insertions, deletions or substitutions of the mutant FGF-1 tends to enhance half-life, which is further enhanced by the inclusion of heparin. Further, mutant heparins can also be used to further enhance the half-life or activity of the mutant FGF-1 used herein. However, heparin is an anti-coagulant that can promote bleeding as a function of increasing concentration. In addition, some individuals have been immunologically sensitized to heparin by previous therapeutic exposure, which can lead to heparin-induced thrombocytopenia and thrombotic events. Mutations that extend the storage stability in vitro and biologic activity in vivo would allow FGF-1 to be formulated and dosed in the absence of exogenous heparin. These include mutations that decrease the rate of oxidative inactivation, such as replacement of one or more of the three cysteine residues by either serine or other compatible residues. In particular, as has been described by others, substitution of cysteine 117 by serine is known to substantially increase the half-life of human FGF-1 by decreasing the rate of oxidative inaction. Other mutations have been described that increase conformational stability by making amino acid changes in internal buried and/or external exposed amino acid residues. In the case of repeat dosing regimens, FGF-1s exhibiting greater stability and life-time might effectively decrease the frequency and number of repeated doses needed to achieve sustained exposure and greater efficacy. These stabilized mutants would allow longer duration dosing from slow release polymeric matrices and delivery systems.

Administration may be performed under fluoroscopy or by other means in order to allow for localization in proximity of the basal ganglia or the substantia nigra. Acceptable carriers, excipients, or stabilizers are also contemplated within the current invention; said carriers, excipients and stabilizers being relatively nontoxic to recipients at the dosages and concentrations employed, and may include buffers such as phosphate, citrate, and other organic acids; antioxidants including ascorbic acid, n-acetylcysteine, alpha tocopherol, and methionine; preservatives such as hexamethonium chloride; octadecyldimethylbenzyl ammonium chloride; benzalkonium chloride; phenol, benzyl alcohol, or butyl; alkyl parabens such as methyl or propyl paraben; catechol; resorcinol; cyclohexinol; 3-pentanol; and mecresol; low molecular weight polypeptides; proteins, such as gelatin, or non-specific immunoglobulins; amino acids such as glycine, glutamine, asparagine, histidine, arginine, or lysine; monosaccharides, disaccharides, and other carbohydrates including glucose, mannose, or dextrins; chelating agents such as EDTA (ethylenediaminetetraacetic acid); sugars such as sucrose, mannitol, trehalose, or sorbitol; and/or salt-forming counter-ions such as sodium. For heparin-binding proteins, including FGFs, heparin may be incorporated into the formulation, which can bind and stabilize the protein against inactivation and degradation.

Surgical Access, Implants, Instruments and Procedures

In many situations, surgical interventions will be required. Once a targeted anatomical region and intended treatment regimen have been determined and where subcutaneous (or deeper) introduction of an angiogenic substance may be desirous, a surgical access path and procedure will typically be determined. In many cases, the simple injection of drugs, proteins, cells and/or compounds into the vasculature and/or soft tissues can be accomplished using hypodermic needles, catheters and/or other minimally- or less-invasive surgical devices. However, where such injections desirably target specific tissues, where such devices may be utilized proximate to sensitive and/or fragile tissues structures, where such devices must transition through and/or into denser or harder tissues, or where a more invasive surgical intervention is desired, additional surgical techniques and/or tools may be required.

In many cases, minimally-invasive devices such as hypodermic needles and cannulae can be introduced via a needle-stick or small incision in the patient's skin and soft tissues and guided to a desired location within the anatomy using fluoroscopic or other non-invasive types of visualization. For example, if minimally-invasive access proximate to a vascular narrowing or blockage is desired, a non-invasive view of the vessel of interest (and surrounding anatomy) may be taken using a fluoroscopic visualization system such as a C-arm, commercially available from GE Medical Systems. The vessel could be visualized on the scan (which may include the use of contrast agent), and the needle tip could be inserted through the patient's skin and soft tissues and advanced until it is proximate to the desired tissue structure(s). It is possible that intraoperative CT, MRI or ultrasound (or other imaging modalities which may or may not yet be in clinical use) may be used by the surgeon to ascertain, to a greater degree of clarity, the exact position of the device and/or verify the location of delivery of the active drug and/or carrier. If the carrier is not radiopaque, then a sufficient amount of a radiopaque material, such as barium powder, may be mixed with the carrier, angiogenic material and/or other injectable compound to allow fluoroscopic visualization and localization of the compound.

In various embodiments described herein, it may be desirous to inject compositions and/or materials, including angiogenic compounds, into specific and/or discrete locations within a patient's anatomy. For example, where imaging, analysis and diagnosis indicates a compromised vascular conduit, it may be desirous to inject an angiogenic factor into and/or near the conduit in an attempt to produce angiogenesis within the localized region. Depending upon the clinical needs, the injection may simply be into the compromised tissues, or the injection may desirably be proximate to the compromised vascular supply (i.e., in tissues adjacent to the vessel constriction and/or obstruction).

If desired, a method of treating a vascular deficiency could include the mechanical creation of a channel or path within various tissues of the patient's body using a hypodermic needle or other device. Once the needle has been advanced along a path, the needle may be withdrawn while concurrently injecting periodic “bursts” (i.e., boli) or a continuous “string” or strings of an angiogenic compound into the path evacuated by the needle, or the injection may comprise a string-like or tube-like structure with FGF-1 infused and/or embedded therein. The desired path may be continuous or intermittent, as desired, and desirably the compound left behind within the path will induce the eventual creation of a new vascular path (or portions thereof) along the needle track.

In various embodiments of the invention, a direct injection of the FGF-1 into and/or adjacent to an ischemic vascular and/or tissue region could be performed to produce and/or induce angiogenesis within a desired region and/or along a desired pathway. In other embodiments, it may be desirous to perform a percutaneous injection (which may include image guided approaches) to access a desired region and/or location within a patient's anatomy. In other embodiments, an open and/or laparoscopic approach may be an optimal approach to a targeted location.

In many cases, it may be desirable to utilize an existing vascular conduit (i.e., a blood vessel) to access a blocked and/or occluded region of the vasculature. Depending upon the location and/or condition of the vessel, the desired angiogenic treatment may be administered within the vessel and/or constriction/blockage, or the angiogenic treatment may be administered outside of the vessel, which may include the employment of a catheter incorporating a deployable needle capable of transiting some or all of the vessel wall (i.e., the needle capable of passing from the inside of the vessel where the catheter resides to the outside of the vessel for medication deployment), which can then desirably inject angiogenic factors in one or more locations in tissues about the periphery of the vessel wall proximate to the occlusion and/or constriction. If desired, such a system could be utilized to transit a first vessel that passes adjacent to a second vessel (the second vessel in need of angiogenic treatment but not in a passable condition for the catheter), wherein the catheter needle exits the first vessel and injects the angiogenic compound proximate to and/or within the second vessel.

In various exemplary embodiments, a hypodermic needle and/or direct catheter can be utilized for injection of therapeutic compounds. This injection can be short term (one injection) or be delivered within an indwelling catheter for longer administration. In addition, a device could be introduced into a targeted location within the pelvic region for longer term introduction of factor(s). If desired, combinations of access routes could be utilized, such as to introduce materials to a plurality of locations and/or to assist with identifying and/or targeting desired anatomical regions—i.e., using a vascular access device within a vessel for identifying the location of an occlusion within a vessel, and a percutaneous approach to inject angiogenic factors outside of, but proximate to, the vessel.

In various embodiments, one or more doses of a therapeutic agent, FGF-1, can be injected directly into one or more ischemic and/or damaged regions of the brain and/or related vascular supply/drainage system, and if such direct injection is not possible, then applied adjacent and/or as closely as possible to the ischemic/damaged tissue regions, which could include injection into tissues and/or vascular channels. One exemplary ideal dose could be determined based on the approximate volume of an ischemic and/or damaged tissue region, such as cell death in the basal ganglia or the substantia nigra, e.g., astrocytes, as estimated using MRI or other imaging modality. If such imaging or assessment were not practical, a clinician could set a standard dose per ischemic/damaged tissue based on an average human tissue volume. In various embodiments, an initial dosing goal for FGF-1 could be to achieve a target concentration of 1 to 10, 50 to 100, 100 to 200, 200 to 300, 300 to 400, 400 to 500, or more ug of FGF-1 per cm³ (˜1 ml) of ischemic/damage tissues. If the specific tissue volume for a given patient can be determined, this value could be converted into dose levels per tissue region or per cm³ of tissue region for each individual patient. For example, one exemplary tissue volume that could potentially be treatable using various aspects of the present invention could include a 1 cm×1 cm×1-2 mm thick volume of damaged and/or ischemic tissues. Alternatively, if an average tissue and/or ischemic/damage tissue volume were determined, a per cm³ dose of such average or actual volume could be used for a patient. In one embodiment, these proposed values could be a dose per treatment day. In other embodiments, efficacy might be improved if weekly or even twice weekly doses were given. For longer term and/or repeated dose treatment of patients, the duration of such long term/repeated dosing could be determined by subsequent MRIs or other imaging of the patient.

Although the exact dosage can be determined on a drug-by-drug basis, in most cases, some generalizations regarding the dosage can be made. The daily small molecule dosage regimen for an adult human patient may be, for example, a dose of between 0.1 mg and 500 mg of FGF-1, preferably between 1 mg and 250 mg, e.g. 5 to 200 mg or a topical, intravenous, subcutaneous, and/or intramuscular dose of each ingredient between 0.01 mg and 100 mg, between 0.1 mg and 60 mg, e.g., 1 to 40 mg of each ingredient of the pharmaceutical compositions disclosed herein or a pharmaceutically acceptable salt thereof calculated as the free base, the composition being administered 1 to 4 times per day. The compositions disclosed herein may be administered by continuous intravenous infusion, preferably at a dose of each ingredient up to 400 mg per day. Thus, in various embodiments the total daily dosage by parenteral administration could typically be in a range 0.1 to 400 mg. In some embodiments, the compounds will be administered for a period of continuous therapy, for example for a week or more, or for months or years. For example, the FGF-1, FGF-1₁₋₁₅₅, FGF-1₁₋₁₄₁, or combinations thereof, is provided at 100, 150, 200, 250, 300, 400, 450, 500, 600, 700, 750, 800, 900 and 1,000 ug/kg/hr to improve for metabolites, behavior, and cell survival in PD. It was found that the treatment provided least one of: an 80, 85, 90, 95, or 100% percent cell survival; an 80, 85, 90, 95, or 100% reduction in PD behaviors, an 80, 85, 90, 95, or 100% survival of the subject, or any combination thereof.

Dosage amount and interval may be adjusted individually to provide plasma levels of the active moiety, which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC (high-performance liquid chromatography) assays or bioassays can be used to determine plasma concentrations. Dosage intervals can also be determined using MEC value. Compositions could be administered using a regimen that maintains plasma levels above the MEC for 10% to 90% of the time, preferably between 30% and 90% and most preferably between 50% and 90%.

The amount of a given composition administered will, of course, be dependent on the subject being treated, on the subject's weight, the severity of the affliction, the manner of administration, the judgment of the prescribing physician, the classification of Parkinson's disease. The extent of impairment with Parkinson's disease includes, bradykinesia, e.g., slowness in initiating voluntary movements, with progressive reduction in speed and range of repetitive actions such as voluntary finger-tapping, in combination with one of three other physical signs: muscular (lead-pipe or cogwheel) rigidity, tremor at rest, and postural instability. Parkinson's disease is described as a synucleinopathy due to an abnormal accumulation of alpha-synuclein protein in the brain.

In various embodiments, it may be desirous to treat an identified deficiency before significant tissue degeneration, damage and/or extent of Parkinson's disease has occurred, even where some portion of the brain vasculature appears to be providing normal blood inflow/outflow, oxygen, nutrition and/or waste removal. This can include imaging and analysis of anatomy proximate to the basal ganglia or the substantia nigra, that can be performed to quantify whether a discrete region and/or tissue should be treated alone or if multiple regions should be treated together. If desired, the imaging data and analysis could provide an ability to compare and contrast various regions to each other (i.e., blood supply of the basal ganglia or the substantia nigra) as well as to compare data over extended periods of time, which could not only help identify tissues at risk, but also identify which vascular and/or tissue elements may be the most contributing to the imbalance for a given patient. In addition, the specific characteristics of the imaging data may demonstrate which vessels and/or tissue architecture may be susceptible to treatment versus other imaging data that shows vascular regions such as capillaries, venules and/or other structures that may be at a stage where treatment may not be as successful. In addition, coupling imaging data with tissue integrity data is used to determine how well the vessels would be predicted to grow into a given area of the basal ganglia or the substantia nigra) and mature into functional vessels capable of providing/removing blood, oxygen, nutrients wastes, enzymes and/or additional NO. Measuring blood inflow and outflow, coupled with analysis of integrity of the basal ganglia or the substantia nigra and 2-D or 3-D mapping of the relevant vasculature might outline the area, level, side and anterior or posterior aspect of the tissues and/or vasculature to be treated. Depending upon the area and structure(s) to be imaged, the data can be reconstructed and/or generated to map the region of interest into right, left, anterior, posterior, caudal and/or cephalad sections for careful analysis of the greatest ischemic and/or hypoxic and/or damaged region(s) of the basal ganglia or the substantia nigra, or relative measures thereof.

In various embodiments, angiogenic treatment may be optimized for use in vascular channels and/or tissues that have significant occlusions and/or calcification, while in other embodiments angiogenic treatment may be optimized for use in vascular channels and/or tissues that do not have significant occlusions and/or calcification. For example, it is believed that calcification and/or other vascular conditions may significantly contribute to the loss of venous compressibility (rendering less effective the desired restriction of blood outflow during tumescence) in the tissues surrounding the basal ganglia or the substantia nigra. However, such deficiencies may only affect localized regions within brain tissues, which may allow an opportunity for a treatment to be utilized that isolates some portion of those deficient regions of venous drainage, desirably allowing less damaged regions of the brain to provide more efficient and/or effective blood outflow control. If desired, angiogenic treatments may be provided that increase the density of astrocytes in such less damaged regions, while vaso-occlusion techniques (such as those described herein) could optionally be utilized to isolate one or more of the damaged regions. Regardless of the frequency of treatment various embodiments described herein include desirably restoring perfusion to the basal ganglia or the substantia nigra (as described herein), which may ultimately provide sufficient blood, oxygen, nutrients and/or waste flow to maintain a minimum or acceptable nutrition level and reverse, reduce and/or slow any degradative cascade of Parkinson's disease.

In various embodiments, angiogenic effects induced in a patient by the various treatments described herein have the potential of creating one or more of the following: (1) a localized improvement in the vasculature and/or microvasculature of the basal ganglia or the substantia nigra and related tissues (or other anatomical locations, including non-extremity areas) proximate to the basal ganglia or the substantia nigra, (2) a systemic or localized improvement by artificially inducing the body to create a collateral flow around an occlusion or blockage in the vasculature of the basal ganglia or the substantia nigra (i.e., artificially inducing a “natural bypass” to the restricted and/or blocked vasculature), (3) a regional or localized improvement in volume of blood flow by artificially inducing the body to create a parallel flow path to existing vasculature in the basal ganglia or the substantia nigra and/or create a new vascular flow path from a source location to a destination location (i.e., a new blood pathway not previously directly connected), (4) a larger volume and/or density of blood vessels in the vascular network leading to the basal ganglia or the substantia nigra, with a commensurate increase in the number and/or density of endothelial cells contained therein (and enzymes and/or other chemicals produced thereby), and/or (5) various combinations thereof. For example, the angiogenic effects of FGF-1 might induce the vasculature and/or capillaries to grow more proximate and/or closer to an area of damaged and/or undernourished tissue (i.e., recruiting blood vessels into previously unperfused/under perfused regions or regions where perfusion has become deficit), which desirably reduces the distance that nutrients and/or oxygen must travel via diffusion into various tissues. In other embodiments, the angiogenic effects might induce the vasculature and/or capillaries to grow more densely in areas proximate and/or closer to a treated region, which could potentially increase the overall availability and/or concentration of enzymes, blood, nutrients and/or oxygen for use by the various vascular channels and/or the basal ganglia or the substantia nigra (which may include tissues located remotely from the more densely grown region). In still other embodiments, the angiogenic effects might induce the vasculature to supplement, repair, bypass and/or reroute a damaged and/or degraded area of vasculature and/or microvasculature, thereby potentially improving localized and/or systemic vascular flow into and/or out of the basal ganglia or the substantia nigra and/or an adjacent anatomical area of the patient's body. In another embodiment, the angiogenic effects might induce the vasculature to open compressed vascular pathways, thereby potentially improving local and/or systemic vascular flow within the basal ganglia or the substantia nigra. In another embodiment, the angiogenic effects might induce growth of the vasculature and/or microvasculature towards healthier and/or larger sources and/or areas of the vasculature (i.e., attaching to and redirecting flow from well-perfused vessels to poorly perfused vessels and/or regions), so as to route additional blood, nutrients and/or oxygen to the treatment area. In another embodiment, the angiogenic effects might induce growth of additional vascular linkages and/or interconnections between various brain tissues. In another embodiment, the angiogenic effects might induce the vasculature to create new pathways that can be compressed, constricted, free from endothelial dysfunction and/or otherwise altered and/or modified by the natural processes of the patient's body in a desired manner, which may be capable of compensating for diseased or damaged anatomy and potentially improving local and/or systemic vascular flow within the basal ganglia or the substantia nigra.

In various embodiments, neuronal effects induced in a patient by the various treatments described herein have the potential of creating one or more of the following: (1) a localized stability and/or improvement in the monoamine and/or metabolite levels of the basal ganglia or the substantia nigra and related tissues (or other anatomical locations, including non-extremity areas) proximate to the basal ganglia or the substantia nigra, (2) a localized improvement in the survival rate of cells in the of the basal ganglia or the substantia nigra and related tissues (or other anatomical locations, including non-extremity areas) proximate to the basal ganglia or the substantia nigra, (3) a regeneration of the tissue that comprises the basal ganglia or the substantia nigra and related tissue, (4) and/or various combinations of the above. For example, the effects of FGF-1 might induce the cells of the substantia nigra or basal ganglia to survive local or systemic conditions that would normally induce apoptosis, autophagic cell death, or programmed necrosis. In another embodiment, the neurological effects of FGF-1 decreases the onset and/or worsening of symptoms related to the death and/or degradation of cells in the basal ganglia or the substantia nigra and related tissues (or other anatomical locations, including non-extremity areas) proximate to the basal ganglia or the substantia nigra. In another embodiment, the neurological effects of FGF-1 induce the tissue of the basal ganglia or the substantia nigra and related tissues (or other anatomical locations, including non-extremity areas) proximate to the basal ganglia or the substantia nigra to proliferate into a more healthy state so that function of the tissue reaches a more natural and/or functional state. In certain embodiments, the improvement is at least a 60, 70, 75, 80, 85, 90, 95, or 100% improvement in monoamine and/or metabolite levels of the basal ganglia or the substantia nigra and related tissues (or other anatomical locations, including non-extremity areas) proximate to the basal ganglia or the substantia nigra, (2) is at least a 60, 70, 75, 80, 85, 90, 95, or 100% a localized improvement in the survival rate of cells in the of the basal ganglia or the substantia nigra and related tissues (or other anatomical locations, including non-extremity areas) proximate to the basal ganglia or the substantia nigra, (3) is at least a 60, 70, 75, 80, 85, 90, 95, or 100% a regeneration of the tissue that comprises the basal ganglia or the substantia nigra and related tissue, (4) and/or various combinations of the above.

It was found that using the present invention, the cell survival rates were improved over those taught previously, in at least three areas. Unlike the prior art, which showed only modest improvements, the present invention led to 60, 70, 75, 80, 85, 90, 95, or 100% survival of the related brain cells and 60, 70, 75, 80, 85, 90, 95, or 100% reduction in behaviors related to PD.

In various embodiments, the medical necessity for angiogenic and/or neuronal treatment can include identifying a patient with early stage Parkinson's disease, correlating a changing quantitative measurement of basal ganglia or substantia nigra degeneration (with either proteoglycan quantification via Tip imaging, ADC, or other quantification techniques) along with diminishing vascular inflow, altered venous outflow and/or increased venous outflow, and optionally assessing the presence and/or absence of vascular calcification and/or other degenerative conditions within the vasculature and/or the basal ganglia or the substantia nigra.

In various embodiments, treatments such as those described herein may be desirous even where a patient has not yet experienced a significant loss of brain function, and/or where a perfusion analysis does not identify a significant hemodynamic imbalance of the brain tissues. Neurodegenerative diseases with a similar pathology could be assigned to the relevant tissue structures, even where MRI of the brain tissues themselves do not directly indicate a vascular mechanism, and/or where x-rays of the anatomy of the brain appears normal. In addition, many new syndromes could potentially be defined by these imaging parameters, including various quantitative measures of vascular health, integrity, metabolism and vascular conditions. In various embodiments, the medical necessity for angiogenic treatment could be based upon various applications and various combinations of these objective data.

In various embodiments, 2D and/or 3D imaging studies could be employed to define the specific and/or localized areas of the vascular circulation and/or brain anatomy that could be best treated with angiogenesis. If one side (left or right) of a blood supply/drainage analysis and/or the basal ganglia or the substantia nigra appeared relatively normal relative to a desired imaging quantifier and/or assessment, and the other side appeared “at risk,” one potential treatment approach could be to provide an angiogenic injection within and/or proximate to the “at risk” area (i.e., injection to only the deficient vasculature and/or the basal ganglia or the substantia nigra). In alternative embodiments, it may be desirous to treat the “normal” or “healthier” area in an attempt to improve perfusion and/or prevent degradation in that level/area, such as where the venous plexus tissues of the basal ganglia or the substantia nigra periphery on one side of the brain are severely calcified and/or non-functional, while those of the “healthier” side have greater functionality. Desirably, a combination of various treatments will desirably restore and/or regenerate tissues of one or both areas (or at least improve such vascularity and/or tissue condition in one or more areas) and produce resulting improvements in perfusion, hemodynamic balance and/or nutrient/waste delivery/removal.

In various embodiments, one or both of the basal ganglia or the substantia nigra could show diminished perfusion in their supply system. It is possible that the basal ganglia or the substantia nigra (and/or the vascular supply and/or drainage thereof) could be treated first and imaging measured for improvement before the other tissue location was treated. Depending upon the level of hemodynamic imbalance, in various embodiments it may be possible for the treatment of the basal ganglia or the substantia nigra to provide sufficient alteration of the brain imbalance to allow a patient to achieve some relief of symptoms.

In one exemplary embodiment, subjects could be scanned using combinations of Magnetic Resonance Imaging (MRI) and Magnetic Resonance Angiography to (MRA) to assess the condition and/or treatability of their pathology. Exemplary 3D Contrast enhanced MRA scans could be acquired with 50 coronal slices using TR:5.1 ms, TE:1.78 ms, voxel size=0.8×0.8×1.5 mm³, SENSE:4. Data acquired in this method could be assessed and/or combined in various ways. For example, the blood supply and/or drainage vessels could be modeled, with the supply and/or drainage vessels on MRA potentially graded as occluded, stenotic or open (or other more graduated assessments could be applied). If desired, relevant tissue locations within the basal ganglia and/or the substantia nigra could be graded. The conditions of the circumferential structures of the basal ganglia and/or the substantia nigra could be analyzed and graded. Image data reflecting the structure and/or perfusion of the various capillary vessels and/or microvasculature in the basal ganglia or the substantia nigra are assessed. In addition, the peripheral branches and/or various arterial blood sources can be analyzed and graded as occluded, stenotic or open (or other more graduated assessments could be applied), and potentially assessed as to whether they could be sufficient to compensate for an ischemic primary blood supply vessel of the basal ganglia or the substantia nigra. In addition, MRI and MRA data sets could be overlaid and/or combined to create composite data maps, including the use of color mapping to identify relevant features of interest, and direct treatment to the basal ganglia or the substantia nigra. In one example, the FGF-1 or mutant FGF-1 is formulated for intramuscularly, intravenously, intra-arterially, or intracerebrally, administration as a solid, gel, liquid, bolus dose, or by infusion from about 0.1 to 100 μg/kg of the patient's body weight, or 0.3 to 30 μg/kg, or 1 to 3 μg/kg of the patient's body weight per dose, and using the imaging described herein, injecting intracerebrally to direct treatment to the site of degradation of the basal ganglia and/or the substantia nigra, e.g., into the nigrostriatal pathway. For example, the FGF-1, FGF-1¹⁻¹⁵⁵, FGF-1¹⁻¹⁴¹, or combinations thereof, is provided at 100, 150, 200, 250, 300, 400, 450, 500, 600, 700, 750, 800, 900 and 1,000 ug/kg/hr to improve for metabolites, behavior, and cell survival in PD. It was found that the treatment provided least one of an 80, 85, 90, 95, or 100% percent cell survival; an 80, 85, 90, 95, or 100% reduction in PD behaviors, an 80, 85, 90, 95, or 100% survival of the subject, or any combination thereof.

Once an ischemic condition and/or other hemodynamic imbalance leading degeneration of the basal ganglia or the substantia nigra has been identified using one or more of the imaging and analysis techniques described herein, various embodiments can include further analysis of anatomical image data of the major circulatory systems that feed into and/or drain out of the brain tissues, to identify any occlusions or partial occlusions (or other pathologies) that may be contributing to the hemodynamic imbalance and/or other causative condition. Where any occlusions or partial occlusions are identified in the imaged regions, a desired course of treatment may include angiogenic and/or surgical treatment of the occlusions or partial occlusions or other abnormalities, alone and/or in combination with other treatments such as angioplasty, stenting, venous occlusion and/or bypass. In some embodiments, if no occlusions or partial occlusions are identified in the supporting vasculature, angiogenic or other treatments may be focused into the basal ganglia or the substantia nigra and/or surrounding brain anatomy.

In addition to FGF-1, the present methods can also use angiogenin, angiopoietin-1, del-1 protein, basic FGF (also known as bFGF or FGF-2), follistatin, granulocyte colony-stimulating factor (G-CSF), hepatocyte growth factor (HGF), interleukin-8 (IL-8), leptin, midkine, placental growth factor, platelet-derived endothelial growth factor (PD-ECGF), platelet-derived growth factor (PDGF), pleiotrophin (PTN), progranulin, proliferin, transforming growth factor alpha (TGF-α), transforming growth factor beta (TGF-β), tumor necrosis factor alpha (TNF-α), vascular endothelial growth factor (VEGF), and vascular permeability factor (VPF). In various embodiments, isolated, recombinant, and/or synthetic angiogenic factors may be used. Angiogenic factors can be administered to a site of ischemic tissue prior to the administration of other materials, concomitant with administration of other materials, or following administration of other materials to the subject.

### Example 1

A FGF-1¹⁵⁻¹⁵⁵ with heparin was tested for efficacy in a mouse Parkinson's disease (PD) model, MPTP (1-methyl-4-phenyl-1,2,3,6-tetrahydropyridine) is a prodrug to the neurotoxin MPP+, which causes permanent symptoms of Parkinson's disease by destroying dopaminergic neurons in the substantia nigra of the brain. In order to determine the efficacy of mutant FGF-1¹⁻¹⁴⁰ with heparin in PD, mice will be injected with MPTP to induce PD and then intravenously with FGF1 at several different doses to determine the influence on attenuation of PD. Intravenous (i.v.) administration of FGF1¹⁻¹⁴⁰ with heparin (0, 5, 10, 50, 100, 200, 500 or 1000 ug/kg) for five days (once per day) will be examined. Administration of FGF1¹⁵⁻¹⁵⁵ with heparin demonstrates a dose dependent attenuation of PD in the mice. Behavioral, biochemical and histological analysis were determined in the mice for FGF-1¹⁵⁻¹⁵⁵ with heparin in PD.

This study was performed to test the efficacy of the human FGF-1¹⁵⁻¹⁵⁵ with heparin in a mouse model of Parkinson's disease (PD). FGF1 is a synthetic human FGF-1 protein. The human FGF-1¹⁵⁻¹⁵⁵ with heparin was provided as a liquid in a 3:1 heparin:FGF1 weight:weight ratio and formulation solution was stored at 4° C.).

Fibroblast growth factor (FGF) exerts a variety of effects on development and maintenance of neurons (Vaccarino et al., 2001; Mufson et al., 1999). Both FGF-1 and FGF-2 are expressed in the brain early in development and this expression persists to adulthood (Vaccarino et al., 1999). While FGF-2 is expressed by both neurons and non-neuronal cells, FGF-1 is primarily expressed in neuronal cells. FGF-1 is found in most neuronal regions (Bean et al., 1991). In addition, FGF receptors (FGFRs) are found in the brain and may mediate signaling initiated by FGFs. FGFs are involved in neurogenesis, axonal growth and branching and in neuroprotection and lesion repair (Vicario-Abejon et al., 1995; Patel and McNamara 1995; Bieger and Unsicker 1995). A number of studies have shown that FGFs are induced following ischemic injury and may play a protective role against neuronal cell death (Ogata et al., 1996). Administration of FGF-1 was shown to protect the gerbil hippocampus from ischemic injury while numerous studies demonstrated the protective effects of FGF-2 in cerebral ischemia (Sasaki et al., 1992; Nakata et al., 1993; Bethel et al., 1997). Clinical trials with FGF-2 did not prove effective possibly due to recruitment of individuals that did not meet the criteria for therapeutic intervention. Because of the lack of treatments for stroke, the identification of compounds that may show protective effects is essential.

Because of the potential role FGF plays in the stroke, FGF-1 was tested to determine its effectiveness for the treatment of PD in a mouse model system. The PD mouse model involves creating PD-like symptoms by i.p. injection of 1-methyl-4-phenyl-1,2,3,6-tetrahydropyridine (MPTP) venous injection. After induction of PD, the human FGF-1¹⁵⁻¹⁵⁵ with heparin (FGF-1) of the present invention was tested for effectiveness in reducing or eliminating the symptoms of PD.

Study design. Male C57BL/6 mice were injected with MPTP as described below and were examined for the protection from PD by FGF-1 at the indicated doses. Animals were examined for behavioral manifestations, biochemical and histological changes.

MPTP treatment. C57BL/6 mice were injected (i.p., 20 mg/kg in 0.1 ml water at 2 hour intervals for 4 doses of 1-methyl-4-phenyl-1,2,3,6-tetrahydropyridine, MPTP, Sigma M-0896) and then examined atone week after injections. Control mice in each group will receive four i.p. injections of saline. Mice were kept on heated blankets for 24 h after the injections.

Administration of FGF-1. For FGF-1 treatment, mice will receive once daily intravenous injections of varying doses of mFGF-1 of 0, 5, 10, 50, 100, 200, 500 or 1000 μg/kg in saline starting 30 min after the MPTP injection and continuing for 7 days, control mice will receive saline only. N=10 per group (5 males and 5 females).

Behavioral testing. Male mice were used in the behavioral testing. All animals were maintained on a 12-h light-dark cycle (lights on at 0700 to 1900) and were permitted free access to food and water.

A 1-h spontaneous activity monitor (vertical and horizontal activity, number of movements over a 5 min period) were conducted via an automated system. To evaluate spontaneous motor activity, we will use an activity monitor consisting of 4 Plexiglas cylinders (23 cm×30 cm, diameter×height) each equipped with three infrared beams and an automated counting system. The spontaneous activity test was started by placing the mouse in the cylinder. After 3 min environmental adaptation, the activity were assessed by counting the number of infrared beam crossings in the photocell apparatus per 5 min.

In addition, to assess sensorimotor coordination, the mice are evaluated for their ability to remain on the rotarod. The rotarod unit consists of a rotating spindle (diameter 7.3 cm) and five individual compartments to test five mice at a time. After twice daily training for two successive days (speed 12 rpm on the first day and 18 rpm on the second day), the rotation speed of test was increased to 25 rpm on the third day in a test session. The test was performed on day 7 and the mice were tested until they were unable to remain on the rotating bar for more than 10 seconds on three consecutive attempts, which are defined as rotarod failure. The time each mouse remains on the rotating bar are recorded for three trials for each mouse, at a 5-min interval and a maximum trial length of 90 s per trial. The time the animals remained on the rotarod (latency) were determined. Data are presented as mean time on the rotating bar over the three test trials.

Beam walking was assessed using 1-m-long wooden beams suspended 80 cm from the floor. The mice were placed on one end of the beam, and the time taken to reach the other end (where the mice entered a cardboard shelter) were assessed. The number of slips and falls in negotiating the beams were recorded. In order to ensure the animals were fully habituated to the test situation, training will take place over 4 days. On day 4 (test day), the mice were given six attempts on the beam. The data presented are the mean of the six attempts. Novelty place preference was assessed in an apparatus with two 29 by 29 by 29-cm compartments, each separated by a small central compartment. Each main compartment was distinguished by color (black or white) and flooring (sandpaper or smooth). During the test, the mouse were placed in one or the other compartment for 60 min, making it familiar relative to the other unexplored and therefore novel side. Then the mice were given a free choice between both compartments for 10 min, and the time spent in the novel side, the number of visits made, and the number of exploratory rears when in the novel side was measured.

Quantification of brain monoamines. Dissected brain regions from animals sacrificed on day 7, was sonicated in 0.1 M perchloric acid and 0.1 mM EDTA (10 mg/100 μl). The extracts were then centrifuged for 15 min and the supernatants were collected and stored at −20° C. Monoamines (noradrenaline, dopamine, serotonin) and metabolites (dihydroxyphenylacetic acid [DOPAC], homovanillic acid [HVA], and 5-hydroxyindolacetic acid [5-HIAA]) were measured with high-pressure liquid chromatography (HPLC) using electrochemical detection.

Immunohistochemistry. All mice were perfused with PBS and 4% PFA. The brains were removed, fixed in 4% PFA for 12 h at 4° C., and then stored in 30% sucrose in PBS. Fifty-micrometer sections were cut and processed for immunohistochemistry using a 1:1000 dilution of a TH antibody (Sigma T-1299). Tyrosine hydroxylase (TH) immunoreactivity was visualized using a monoclonal anti-TH antibody. Preliminary quantification of TH-immunopositive cells in the substantia nigra and ventral tegmental areas were made using image analysis. Sections were dried and mounted in Depex. Cell counting was performed using a computer-assisted stereological toolbox. All cell counts were done blind to drug treatments and performed at 100-fold magnification.

Statistical analysis. The results were expressed as the mean standard deviation (SD). The significance of difference in the data were analyzed using a t-test.

Exclusion of Animals From the Study. Animals were excluded from the study based upon several criteria:
1. Animals that died prior to completion of study (at any point).
2. Animals developed severe complications following administration of test articles.

Treatment groups. All groups were subjected to FGF-1 or were controls. Animals (60 animals) were subjected to bolus i.v. dosing by tail vein of vehicle or FGF-1 at the indicated doses. Animals were injected 1 time per day for 5 days.

Mouse MS Model:

| Group | | | |
|---|---|---|---|
| C57BL/6 male mice | Compound | Dose (μg/kg) | Route |
| 1 (n = 10 mice) | Sham | 0 | IV |
| 2 (n = 10 mice) | PD Control | 0 | IV |
| 3 (n = 10 mice) | mFGF-1 | 5 ug/kg/day | IV |
| 4 (n = 10 mice) | mFGF-1 | 10 ug/kg/day | IV |
| 5 (n = 10 mice) | mFGF-1 | 50 ug/kg/day | IV |
| 6 (n = 10 mice) | mFGF-1 | 100 ug/kg/day | IV |
| 7 (n = 10 mice) | mFGF-1 | 200 ug/kg/day | IV |
| 8 (n = 10 mice) | mFGF-1 | 500 ug/kg/day | IV |
| 9 (n = 10 mice) | mFGF-1 | 1000 ug/kg/day | IV |

Endpoints. Modulation of PD in the mouse. FGF-1 with heparin (FGF-1) was provided in a liquid buffer. All animals in the test groups were dosed as indicated above.

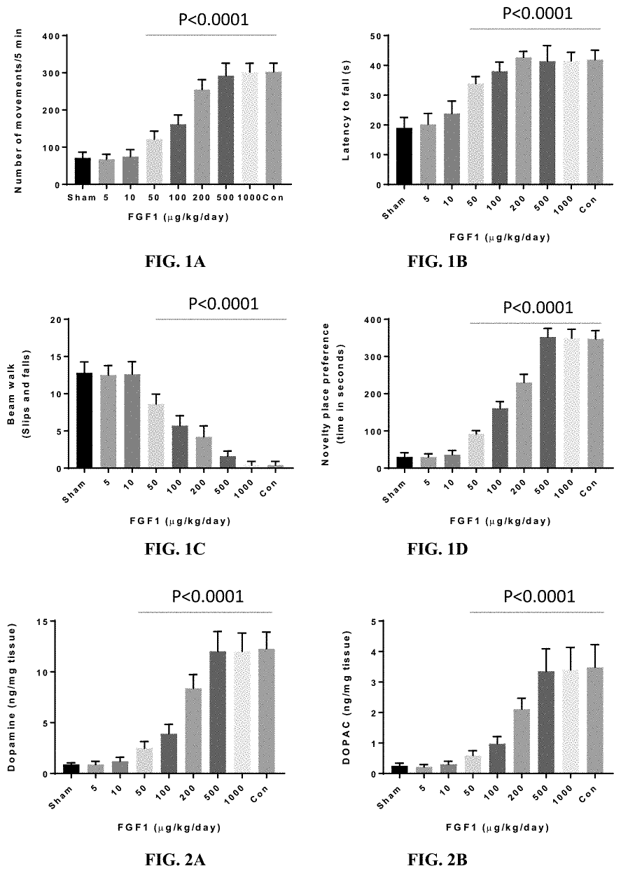

Behavioral analysis. The efficacy of FGF-1 in a mouse model of MS was assessed. Data from mice that were i.v. administered with vehicle or FGF-1 (at indicated doses). After induction of PD with MPTP, the mice were administered FGF-1 at the indicated doses (FIGS. 1A-1D and Tables 1 and 2). Animals were examined on day 2 and every day to determine the behavior of the animals following MPTP and FGF-1. Mice were injected with FGF-1 every day for five days starting on the day of MPTP administration. The FGF-1 was started 30 min after the last MPTP injection and continued for four additional days. As seen in FIGS. 1A-1D, the mice treated with vehicle showed a significant decrease in behavioral scores (spontaneous activity, rotarod tests, beam walk and novel place preference) compared to the control or treated animals. Treatment with FGF-1 showed a significant improvement (attenuation) in the behavior (FIGS. 1A-1D and Tables 1-4). FGF-lat 50 to 1000 μg/kg, showed a significant benefit, whereas the 5-10 μg/kg did not show any improvement.

TABLE 1
Spontaneous locomotor activity

| Treatment | Number of movements/5 min |
|---|---|
| Vehicle (Sham) | 71.30 ± 15.12 (NA) |
| FGF1 (5 μg/kg/day) | 67.60 ± 13.28 (0.5683) |
| FGF1 (10 μg/kg/day) | 74.50 ± 19.12 (0.6829) |
| FGF1 (50 μg/kg/day) | 121.60 ± 21.69 (<0.0001) |
| FGF1 (100 μg/kg/day) | 161.50 ± 24.95 (<0.0001) |
| FGF1 (200 μg/kg/day) | 254.90 ± 26.69 (<0.0001) |
| FGF1 (500 μg/kg/day) | 292.00 ± 33.75 (<0.0001) |
| FGF1 (1000 μg/kg/day) | 301.90 ± 23.68 (<0.0001) |
| Control | 302.40 ± 23.37 (<0.0001) |

Mean ± SD

TABLE 2
Rotarod Test -

| Treatment | Latent Period |
|---|---|
| Vehicle (Sham) | 19.0 ± 3.528 (NA) |
| FGF1 (5 μg/kg/day) | 20.2 ± 3.645 (0.4641) |
| FGF1 (10 μg/kg/day) | 23.8 ± 4.237 (0.0131) |
| FGF1 (50 μg/kg/day) | 33.9 ± 2.331 (<0.0001) |
| FGF1 (100 μg/kg/day) | 38.0 ± 3.091 (<0.0001) |
| FGF1 (200 μg/kg/day) | 42.7 ± 2.003 (<0.0001) |
| FGF1 (500 μg/kg/day) | 41.4 ± 5.254 (<0.0001) |
| FGF1 (1000 μg/kg/day) | 41.5 ± 2.877 (<0.0001) |
| Control | 41.9 ± 3.213 (<0.0001) |

Mean ± SD

TABLE 3
Beam Walk

| Treatment | Slips and falls |
|---|---|
| Vehicle (Sham) | 12.8 ± 1.476 (NA) |
| FGF1 (5 μg/kg/day) | 12.5 ± 1.269 (0.6319) |
| FGF1 (10 μg/kg/day) | 12.6 ± 1.713 (0.7829) |
| FGF1 (50 μg/kg/day) | 8.6 ± 1.35 (<0.0001) |
| FGF1 (100 μg/kg/day) | 5.7 ± 1.337 (<0.0001) |
| FGF1 (200 μg/kg/day) | 4.2 ± 1.476 (<0.0001) |
| FGF1 (500 μg/kg/day) | 1.6 ± 0.6992 (<0.0001) |
| FGF1 (1000 μg/kg/day) | 0.4 ± 0.5164 (<0.0001) |
| Control | 0.4 ± 0.5164 (<0.0001) |

Mean ± SD

TABLE 4
Novel place preference

| Treatment | Slips and falls |
|---|---|
| Vehicle (Sham) | 30.3 ± 11.31 (NA) |
| FGF1 (5 μg/kg/day) | 29.9 ± 8.647 (0.9302) |
| FGF1 (10 μg/kg/day) | 36.1 ± 11.4 (0.2684) |
| FGF1 (50 μg/kg/day) | 91.7 ± 8.97 (<0.0001) |
| FGF1 (100 μg/kg/day) | 160.7 ± 18.17 (<0.0001) |
| FGF1 (200 μg/kg/day) | 230.0 ± 22.25 (<0.0001) |
| FGF1 (500 μg/kg/day) | 352.6 ± 22.89 (<0.0001) |
| FGF1 (1000 μg/kg/day) | 349.0 ± 24.060 (<0.0001) |
| Control | 347.0 ± 22.53 (<0.0001) |

Mean ± SD

Mortality: There were no deaths in this study.

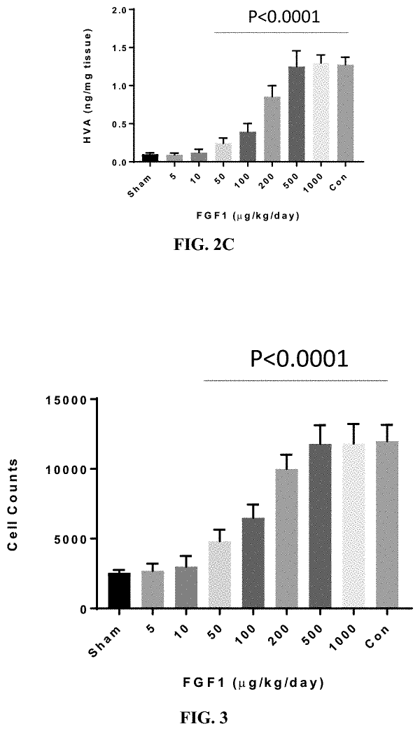

Monoamine and metabolite levels in the brains of MPTP treated animals. The efficacy of FGF-1 on brain monoamine and metabolite levels that change during treatment with MPTP and are markers of PD were determined. As seen in Table 5 and FIGS. 2A-2C, the levels of dopamine, DOPAC and HVA all were higher in the FGF-1 treated animals compared to the vehicle treated animals. The 200-500 μg/kg/day showed the greatest protection following MPTP treatment.

TABLE 5
Monoamine and metabolite levels

| | | Levels | | |
|---|---|---|---|---|
| | Dose | (ng/mg tissue) | | % |
| Region | (μg/kg) | Mean +/− SD | P value | difference |
| Dopamine | 0 (Sham) | 0.891 ± 0.1497 | NA | NA |
| | 5 | 0.883 ± 0.3168 | 0.9192 | |
| | 10 | 1.202 ± 0.391 | 0.0020 | |
| | 50 | 2.497 ± 0.6475 | <0.0001 | |
| | 100 | 3.908 ± 0.9202 | <0.0001 | |
| | 200 | 8.386 ± 1.344 | <0.0001 | |
| | 500 | 12.02 ± 1.943 | <0.0001 | |
| | 1000 | 12.01 ± 1.811 | <0.0001 | |
| | Control | 12.27 ± 1.655 | <0.0001 | |
| DOPAC | 0 | 0.251 ± 0.0902 | NA | NA |
| | 5 | 0.2145 ± 0.8036 | 0.1846 | |
| | 10 | 0.303 ± 0.09937 | 0.0912 | |
| | 50 | 0.5915 ± 0.1579 | <0.0001 | |
| | 100 | 0.974 ± 0.2344 | <0.0001 | |
| | 200 | 2.109 ± 0.3608 | <0.0001 | |
| | 500 | 3.354 ± 0.7365 | <0.0001 | |
| | 1000 | 3.397 ± 0.7378 | <0.0001 | |
| | Control | 3.48 ± 0.7481 | <0.0001 | |
| HVA | 0 | 0.0995 ± 0.01726 | NA | NA |
| | 5 | 0.0925 ± 0.02219 | 0.3601 | |
| | 10 | 0.1214 ± 0.04228 | 0.0840 | |
| | 50 | 0.2429 ± 0.07.32 | <0.0001 | |
| | 100 | 0.3943 ± 0.108 | <0.0001 | |
| | 200 | 0.8536 ± 0.1455 | <0.0001 | |
| | 500 | 1.249 ± 0.2088 | <0.0001 | |
| | 1000 | 1.296 ± 0.1059 | <0.0001 | |
| | Control | 1.274 ± 0.09756 | <0.0001 | |

Cell Counts. Following the behavioral studies, the animals were sacrificed at 7 days and ½ of the brain was taken and stained for tyrosine hydroxylase (TH) positive neurons in the substantia nigra pars compacta (SNpc). MPTP selectively kills these neurons and this is an excellent marker to determine the effect of FGF-1 on attenuation of the disease. The numbers of cells in the SNpc were determined by counting the TH positive neurons and are shown in Table 6 and FIG. 3. As the dose of FGF-1 increased, cell loss was prevented and the number of cells increased in the brain of the FGF-1 treated mice. This demonstrates that FGF-1 protects the brain from the detrimental effects of PD induction.

TABLE 6
Cell counts

| | | Cell Counts | | |
|---|---|---|---|---|
| | Dose | Mean +/− SD | | % |
| Region | (ug/kg) | (#/50 μm) | P value | difference |
| SNpc | 0 | 2555 ± 206.8 | NA | NA |
| | 5 | 2698 ± 510.6 | 0.4218 | |
| | 10 | 2988 ± 763.3 | 0.1004 | |
| | 50 | 4820 ± 823.2 | <0.0001 | |
| | 100 | 6494 ± 944.1 | <0.0001 | |
| | 200 | 9993 ± 1025 | <0.0001 | |
| | 500 | 11797 ± 1339 | <0.0001 | |
| | 1000 | 11806 ± 1410 | <0.0001 | |
| | Control | 11990 ± 1169 | <0.0001 | |

FGF-1 is a trophic factor that may provide protection from neurological diseases and allow for regeneration of neuronal tissue following injury or grafting. The studies performed here demonstrate the efficacy of the FGF-1 to attenuate PD in a mouse model. Intravenous administration of FGF-1 at 50 to 1000 ug/kg bolus dose over a five-day period demonstrated a dose dependent decrease in PD behavior, biochemistry and histology anomaly. This shows that FGF-1 is effective in limiting the extent of PD in the mouse via intravenous administration and may be beneficial for treating this disease.

When administered intravenously, FGF-1 was found to be efficacious in a mouse model of PD. The effectiveness of FGF-1 was dose dependent and indicates that FGF-1 is beneficial in PD treatment.

In one embodiment, the present invention includes a method of treating Parkinson's disease in a patient consisting essentially of, or consisting of: administering a therapeutically effective amount of a FGF-1 conjugated to a heparin (FGF-1), which is an amount sufficient to reduce or eliminate one or more symptoms associated with Parkinson's disease. In one aspect, the FGF-1 is selected from 140 or 141 amino acid protein of SEQ ID NO:1, which is the mature form of human, also referred to as: FGF-1¹⁻¹⁴⁰, FGF-1¹⁻¹⁴¹, FGF-1¹⁰⁻¹⁴⁰, FGF-1¹⁰⁻¹⁴¹, FGF-1¹⁻¹⁴⁰, FGF-1¹²⁻¹⁴⁰, FGF-1¹²⁻¹⁴¹, and mature FGF-1 with point mutations including, for example, one or more of the following: K9A, K12V, S17R, N18R, N18K, H21Y, R35E, L44F, A66C, Y94V, N95V, H102Y, F108Y, N114R, N114K, C117V, L133A of SEQ ID NO:1.

In another embodiment, the present invention includes a method treating Parkinson's disease in a patient consisting essentially of, or consisting of: imaging a patient to generate image data; obtaining imaging data of at least a portion of a microvasculature region of the patient proximal to a portion of a brain proximal to a basal ganglia or a substantia nigra; assessing the imaging data to quantify localized perfusion of at least a portion of the microvasculature proximal to the basal ganglia or the substantia nigra and identify at least one hypoperfused region of the microvasculature; and treating the at least one hypoperfused region of the microvasculature by introducing a first amount of a compound comprising FGF-1 in a scaffold material proximate to the hypoperfused region of the microvasculature at the basal ganglia or the substantia nigra, wherein the FGF-1 is administered for extended released. In one aspect, the FGF-1 is selected from 140 or 141 amino acid protein of SEQ ID NO:1, which is the mature form of human, also referred to as: FGF-1¹⁻¹⁴⁰ FGF-1¹⁻¹⁴¹ FGF-1¹⁰⁻¹⁴⁰ FGF-1¹⁰⁻¹⁴¹, FGF-1¹⁻¹⁴⁰, FGF-1¹²⁻¹⁴⁰, FGF-1¹²⁻¹⁴¹, and mature FGF-1 with point mutations including, for example, one or more of the following: K9A, K12V, S17R, N18R, N18K, H21Y, R35E, L44F, A66C, Y94V, N95V, H102Y, F108Y, N114R, N114K, C117V, L133A of SEQ ID NO:1.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

What is claimed is:

1. A method of treating Parkinson's disease in a patient comprising:
administering a therapeutically effective amount of a Fibroblast Growth Factor-1 (FGF-1), or active mutants or variants thereof, conjugated to a heparin (FGF-1), which is an amount sufficient to reduce or eliminate one or more symptoms associated with Parkinson's disease.

2. The method of claim 1, wherein the active mutant or variant FGF-1 is selected from 140 or 141 amino acid protein of SEQ ID NO:1, which is the mature form of human, also referred to as: FGF-1¹⁻¹⁴⁰, FGF-1¹⁻¹⁴¹, FGF-1¹⁰⁻¹⁴⁰, FGF-1¹⁰⁻¹⁴¹, FGF-1¹⁻¹⁴⁰, FGF-1¹²⁻¹⁴⁰, FGF-1¹²⁻¹⁴¹, and mature FGF-1 with point mutations including, for example, one or more of the following: K9A, K12V, S17R, N18R, N18K, H21Y, R35E, L44F, A66C, Y94V, N95V, H102Y, F108Y, N114R, Ni14K, C117V, L133A of SEQ ID NO:1.

3. The method of claim 1, wherein the FGF-1 is formulated for intravenous administration.

4. The method of claim 1, further comprising the steps of:
imaging a patient to generate image data;
obtaining image data of the patient, the image data including at least a portion of a brain proximal to a basal ganglia or a substantia nigra; and
introducing at or about the basal ganglia or the substantia nigra a first amount of a FGF-1.

5. The method of claim 1, further comprising introducing a first therapeutically effective amount of at least one member of the group consisting of embryonic stem cells, adult stem cells, stem cells and progenitor cells to a brain proximal to, or into, a basal ganglia or a substantia nigra.

6. The method of claim 5, wherein the step of injecting the therapeutically effective amount of at least one member of the group consisting of embryonic stem cells, adult stem cells, stem cells and progenitor cells into a portion of a brain proximal to a basal ganglia or a substantia nigra via a percutaneous route.

7. The method of claim 1, further comprising surgically implanting a tissue graft proximate to a portion of a brain proximal to a basal ganglia or a substantia nigra with an amount the FGF-1, active mutants or variants sufficient to reduce or eliminate the symptoms of PD, wherein the tissue graft comprises at least one member of the group consisting of embryonic stem cells, adult stem cells, stem cells and progenitor cells into the basal ganglia or the substantia nigra after the tissue graft has been surgically implanted.

8. The method of claim 1, further comprising the steps of obtaining non-invasive image data of the subject after treatment with the FGF-1, wherein the non-invasive image data including the at-risk region of a brain, and analyzing the non-invasive image data to identify any improvement in the blood vessel supply to the at-risk region of the brain.

9. The method of claim 1, wherein the FGF-1 further comprises a time release, biodegradable carrier applied at a surgical grafting site proximal to the basal ganglia or the substantia nigra.

10. The method of claim 1, wherein the FGF-1 is administered from about 0.5 to 1000 μg/kg, or 1 to 500 μg/kg, or 10 to 500 μg/kg, or 50 to 100 μg/kg of the patient's body weight per dose.

11. The method of claim 1, wherein the FGF-1 is formulated for intramuscularly, intravenously, intra-arterially, or intracerebrally, administration as a solid, gel, liquid, bolus dose, or by infusion from about 0.1 to 100 μg/kg of the patient's body weight, or 0.3 to 30 μg/kg, or 1 to 3 μg/kg of the patient's body weight per dose.

12. The method of claim 1, wherein the FGF-1 is not administered intranasally, intraperitoneally, or traversing the cranium.

13. The method of claim 1, wherein the dose of FGF-1 is greater than 100 ng/ml.

14. The method of claim 1, wherein the FGF-1 reduces Parkinson's progression without triggering angiogenesis.

15. The method of claim 1, wherein the FGF-1, FGF-1₁₋₁₅₅, FGF-1₁₋₁₄₁, or combinations thereof, is provided at 100, 150, 200, 250, 300, 400, 450, 500, 600, 700, 750, 800, 900 and 1,000 ug/kg/hr to improve for metabolites, behavior, and cell survival in PD.

16. The method of claim 1, wherein the treatment provides least one of: an 80, 85, 90, 95, or 100% percent cell survival; an 80, 85, 90, 95, or 100% reduction in PD behaviors, an 80, 85, 90, 95, or 100% survival of the subject, or any combination thereof.

17. A method treating Parkinson's disease in a patient comprising the steps of:
imaging a patient to generate image data;
obtaining imaging data of at least a portion of a microvasculature region of the patient proximal to a portion of a brain proximal to a basal ganglia or a substantia nigra;
assessing the imaging data to quantify localized perfusion of at least a portion of the microvasculature proximal to the basal ganglia or the substantia nigra and identify at least one hypoperfused region of the microvasculature; and
treating the at least one hypoperfused region of the microvasculature by introducing a first amount of a compound comprising a Fibroblast Growth Factor-1 (FGF-1), or active mutants or variants thereof in a scaffold material proximate to the hypoperfused region of the microvasculature at the basal ganglia or the substantia nigra, wherein the FGF-1 is administered for extended released.

18. The method of claim 17, wherein the active mutant or variant FGF-1 is selected from 140 or 141 amino acid protein of SEQ ID NO:1, which is the mature form of human, also referred to as: FGF-1¹⁻¹⁴⁰, FGF-1¹⁻¹⁴¹, FGF-1¹⁰⁻¹⁴⁰, FGF-1¹⁰⁻¹⁴¹, FGF-1¹⁻¹⁴⁰, FGF-1¹²⁻¹⁴⁰, FGF-1¹²⁻¹⁴¹, and mature FGF-1 with point mutants including, for example, one or more of the following: K9A, K12V, S17R, N18R, N18K, H21Y, R35E, L44F, A66C, Y94V, N95V, H102Y, F108Y, N114R, N114K, C117V, L133A of SEQ ID NO:1.

19. The method of claim 17, wherein the FGF-1 is formulated for intravenous administration.

20. The method of claim 17, further comprising the steps of:
imaging a patient to generate image data;
obtaining image data of the patient, the image data including at least a portion of a brain proximal to a basal ganglia or a substantia nigra; and
introducing at or about the basal ganglia or the substantia nigra a first amount of the FGF-1.

21. The method of claim 17, further comprising introducing a first therapeutically effective amount of at least one member of the group consisting of embryonic stem cells, adult stem cells, stem cells and progenitor cells to a brain proximal to, or into, a basal ganglia or a substantia nigra.

22. The method of claim 21, wherein the step of injecting the therapeutically effective amount of at least one member of the group consisting of embryonic stem cells, adult stem cells, stem cells and progenitor cells into a portion of a brain proximal to a basal ganglia or a substantia nigra via a percutaneous route.

23. The method of claim 17, further comprising surgically implanting a tissue graft proximate to a portion of a brain proximal to a basal ganglia or a substantia nigra with an amount FGF-1 sufficient to reduce or eliminate the symptoms of PD, wherein the tissue graft comprises at least one member of the group consisting of embryonic stem cells, adult stem cells, stem cells and progenitor cells into the basal ganglia or the substantia nigra after the tissue graft has been surgically implanted.

24. The method of claim 17, further comprising the steps of obtaining non-invasive image data of the subject after treatment with the FGF-1, wherein the non-invasive image data including the at-risk region of a brain, and analyzing the non-invasive image data to identify any improvement in the blood vessel supply to the at-risk region of the brain.

25. The method of claim 17, wherein the FGF-1 further comprises a time release, biodegradable carrier applied at a surgical grafting site proximal to the basal ganglia or the substantia nigra.

26. The method of claim 17, wherein the FGF-1 is administered from about 0.5 to 1000 μg/kg, or 1 to 500 μg/kg, or 10 to 500 μg/kg, or 50 to 100 μg/kg of the patient's body weight per dose.

27. The method of claim 17, wherein the FGF-1 is formulated for intramuscularly, intravenously, intracranially, or intra-arterially, administration as a solid, gel, liquid, bolus dose, or by infusion from about 0.1 to 100 μg/kg of the patient's body weight, or 0.3 to 30 μg/kg, or 1 to 3 μg/kg of the patient's body weight per dose.

28. The method of claim 17, wherein the FGF-1 is not administered intranasally, intraperitoneally, or traversing the cranium.

29. The method of claim 17, wherein the dose of FGF-1 is greater than 100 ng/ml.

30. The method of claim 17, wherein the FGF-1 reduces Parkinson's progression without triggering or causing angiogenesis.

31. The method of claim 17, wherein the FGF-1, FGF-1₁₋₁₅₅, FGF-1₁₋₁₄₁, or combinations thereof, is provided at 100, 150, 200, 250, 300, 400, 450, 500, 600, 700, 750, 800, 900 and 1,000 ug/kg/hr to improve for metabolites, behavior, and cell survival in PD.

32. The method of claim 17, wherein the treatment provides least one of: an 80, 85, 90, 95, or 100% percent cell survival; an 80, 85, 90, 95, or 100% reduction in PD behaviors, an 80, 85, 90, 95, or 100% survival of the subject, or any combination thereof.


SEQUENCE LISTING (2 sequences)

SEQ ID NO: 1 - protein, 141 aa, Artificial Sequence
  Synthetic peptide
MFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 2 - protein, 155 aa, Artificial Sequence
  Synthetic peptide
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD